(19) United States
(12) Patent Application Publication
(10) Pub. No.: US 2011/0124517 A1
(43) Pub. Date: May 26, 2011

(54) Process for Determining the Genotype from a Biological Sample Containing Nucleic Acids of Different Individuals

(75) Inventors: Christoph GAUER, Muenchen (DE); Wolfgang MANN, Neudrossenfeld (DE)

(73) Assignee: BECKMAN COULTER, INC., Brea, CA (US)

(21) Appl. No.: 12/086,370

(22) PCT Filed: Oct. 24, 2006
(86) PCT No.: PCT/EP2006/010245

§ 371 Date: Jun. 9, 2010

(30) Foreign Application Priority Data
Dec. 12, 2005 (DE) 10 2005 059 227.9

(51) Int. Cl. C40B 30/04; C12Q 1/68

(52) U.S. Cl. 506/9; 435/6

(57) ABSTRACT

The present Invention relates to a process for determining the genotype of one or more individuals from a biological sample which contains nucleic acids from different individuals, in particular a process for determining the number of copies of a predetermined sequence, in which first using at least two subquantities of the biological sample of different concentrations, in each case at least one amplification reaction is carried out, subsequently the number of the different amplification products obtained for each of the at least two subquantities is determined and compared with one another, and finally the amplification products which were obtained only for one defined subquantity and/or those amplification products which were obtained for all subquantities, are characterized. In addition, the present invention relates to a kit for carrying out the process according to the invention.

The present invention relates to a method for the determination of the genotype of one or more individuals from a biological sample which contains nucleic acids of different individuals, in particular a method for determining the copy number of a predetermined sequence and also a kit for the determination of the genotype of one or more individuals from a biological sample containing nucleic acids of different individuals.

Methods for the determination of the genotype of individuals from a biological sample, i.e. for example the proof of the presence or of the absence of specific sequences, such as for example of individual genes or of gene sections or the determination of the quantity of specific nucleic acids, are used in many technical fields. Simply by way of example applications in forensic science, gene technology, for example in the context of cloning, or in medical diagnostics should be named.

A technique for the characterization of the genotype of one or more individuals frequently used in criminal science, forensic science or in determinations of fatherhood or relatives is the generation of a genetic finger-print. For the generation of a genetic fingerprint two methods are in particular used nowadays, namely on the one hand the RFLP technique (restriction fragment length polymorphism technique) and also the VNTR typification (variable numbers of tandem repeats typification). In both methods DNA is first isolated from biological trace material which for example contains blood, saliva, hairs with roots, sperm or vaginal secretion. After the isolation of the DNA from the biological sample the DNA is hybridized in the RFLP method with the aid of restriction enzymes into a plurality of DNA portions of different length before the individual DNA fragments are separated in accordance with their length on an Agarose gel. Subsequently the individual DNA fragments are transferred with the aid of the known southern blot methods from the Agarose gel onto a nylon membrane and are fixed on the membrane. The nylon membrane is subsequently hybridized with specific fluorescence marked probes in order to show the existence of specific DNA fragments. Finally the DNA fragments hybridized with the specific probes are visualized for example by enzyme reaction in order to obtain a band pattern called a genetic fingerprint. Conclusions regarding the genotype of the individual can be drawn by the relative position of the individual bands, for example by comparison with the corresponding patterns of a reference sample. However, for the carrying out of the RFLP technique very large quantities of DNA are required so that this method is nowadays solely used for determinations of the fatherhood or relatives of living persons, because only in these applications is sufficient DNA available. For forensic methods such as for example solving violent crimes in which often only very small traces of DNA are available the RFLP method is not, however, suitable. Moreover, the RFLP method only delivers reliable values when the biological starting sample only contains DNA of one individual.

In the VNTR typification selected DNA ranges from the non-coded region of the genome are multiplied by PCR (polymerase chain reaction) before the amplified DNA fragments are separated in accordance with their length, for example on a polyacrylamid gel and are subsequently visualized. Conclusions can be drawn with a certain probability from the so obtained length pattern by comparison with the band pattern obtained with the corresponding reference sample, regarding the identity or non-identity of the donor of the reference sample with the individual whose nucleic acid is present in the biological sample. A disadvantage of this method is, however, that it is relatively time-consuming, in particular with regard to the separation of the DNA fragments on a polyacrylamid gel. Moreover, this method only delivers reliable results when the DNA of one individual is contained in the investigated biological sample. If the biological starting sample however contains nucleic acid of different individuals, the VNTR typification fails in just the same way as the RFLP technique.

In molecular diagnostics methods for the characterization of the genotype of an individual, for example for the quantification of sequences, in particular for the quantitative determination of the number of copies of nucleic acid sequences, are gaining an ever more important role. Since a multitude of partly serious illnesses are caused by deviations from the normal number of copies of nucleic acid sequences in the genome, a determination of the number of copies of specific chromosomes or of specific gene sections makes it possible to diagnose corresponding illnesses reliably even at an early stage of the development.

Examples for partly serious anomalies which can be attributed to an increased number of copies of whole chromosomes are trisomy 18 (Edward's syndrome), trisomy 13 (Pätau syndrome) and also trisomy 21 (Down syndrome). For each of these illnesses the number of copies of the corresponding chromosomes 18, 13 and 21 is three per cell whereas healthy individuals only have two copies of the above-named chromosomes per cell. In all three cases the increase of the number of copies of the relevant chromosomes leads to most serious development problems. Whereas carriers of the trisomy 21 are severely handicapped in their development and partly have serious deformities, the carriers of trisomy 18 and trisomy 13 mainly die within the first year of life.

In addition to illnesses which can be attributed to an increased number of copies of whole chromosomes there is also a multitude of illnesses which are known which relate to a changed number of copies of genes or gene sections. Simply by way of example, the Huntington disease should be named in this connection, a progressively developing neuro-degenerative illness characterized by abnormal involuntary movements with increasing deterioration of the mental and physical capabilities.

As a result of the requirement for methods for the quantification of sequence copies in a biological sample, a number of corresponding methods was proposed in the past.

One of the basic quantification methods which permit at least a statement concerning the presence or absence of nucleic acid sequences and, depending on how the method is carried out, also a conditional conclusion on the number of copies of the relevant nucleic acid sequences per cell, is the so-called FISH-method (fluorescence in situ hybridization). In this method the biological sample to be investigated is incubated after appropriate pre-treatment with one or more different probes which were previously marked with respectively different fluorescent dyes under conditions which enable a hybridization of the probes with sequences homologous thereto in the biological sample. After the hybridization the samples are washed, with non-specific hybridization signals being eliminated. Finally the fluorescence signals of the preparation are evaluated with a fluorescence microscope. Each fluorescence signal that is present points to the presence of the sequence corresponding to the probe provided with the corresponding fluorescent marker. The intensity of the fluorescence can allow a conditional conclusion to be drawn on the number of the sequence copies in the biological sample. A disadvantage of the named method lies in the fact that an undesired cross-hybridization which leads to incorrect results can never be fully precluded. Moreover, this method is comparalively expensive because, on the one hand, fluorescent dyes must necessarily be used and, on the other hand, because it requires complicated apparatuses, such as fluorescence microscopes. Furthermore, the ability of this method to produce reliable results depends quite decisively on the quality of the probes that are used; reliable results are only obtained when the probes hybridize with an effectiveness of more than 90% onto the binding positions corresponding thereto, so that only 10% of the target sequences are present in non-hybridized form and are as a consequence not detected. Finally, this method does not deliver reliable results when a biological sample is used which contains nucleic acids of different individuals.

Another fluorescent-based method is the CGH-analysis (comparative genomic hybridization). In this method the nucleic acid of the sample to be analyzed is completely marked with a dye 1. The same quantity of nucleic acid of a reference sample is marked with a dye 2. The two reaction batches are jointly hybridized to a spread metaphase chromosome set, with the sequences contained in the two reaction batches competing for the binding sites to the spread chromosomes. Essentially a ratio of dye 1 to dye 2 such as 1:1 arises at all hybridization points. If the sample to be analyzed contains amplified regions (more than the usual copy number of the reference) then the dye 1 will predominate at this hybridization point. In the event of a deletion in the sample to be investigated one will only detect the dye 2 at this hybridization point. The reference measurement permits a relative statement concerning the frequency of sequences in the sample to be analyzed.

The method is complicated and expensive so that the technical apparatus which are required must be very accurately set and/or calibrated; two dyes with different marking are required and the standardization of these experiments is time-consuming and difficult. Moreover, this method does not deliver any exploitable results when a biological sample is used which contains nucleic acid of different individuals.

A further known method for the quantification of nucleic acid sequences is the real-time-PCR-method in which a PCR is, for example, carried out with fluorescence-marked primers and the increase of the fluorescence signal in dependence on the number of cycles is observed. The threshold value PCR-cycle (also threshold-cycle) is associated with the reaction time point at which the fluorescence signal is significantly distinguished from the background fluorescence and the PCR product formation runs exponentially. This correlates with the starting copy number of the DNA sequence to be augmented. In this manner DNA samples can be quantified relatively with respect to the comparison with a DNA dilution series. A disadvantage of this method however lies in the fact that the quantity of starting material cannot be reduced in size arbitrarily because, with a few starting molecules, for example 10 to 100 copies as a starting material, the stochastic error becomes very large as a result of the exponential amplification which no longer permits a quantitative statement. Furthermore, this method requires complicated and expensive apparatuses for the measurement of the fluorescence intensity. Finally, this method also fails when a biological sample is used which contains nucleic acids of different individuals.

All the above-named methods are based on the use of fluorescent dyes and require expensive apparatuses for the determination of the intensity of fluorescence. Moreover, these require the use of a minimum quantity of starting material in order to obtain results which are at least somewhat reliable. Moreover, none of the above-named methods is suitable for determining the genotype of an individual from a biological sample which contains nucleic acids of different individuals.

The object of the present invention is thus to make available a method for the determination of the genotype of one or more individuals from a biological sample which contains nucleic acids of different individuals and which is in particular suitable for the determination of the copy number of a predetermined sequence and of sequences homologous thereto, for example the absolute or relative number of alleles in biological sample, which is moreover simple and cost-favorable to carry out and which also delivers reliable results, in particular even with small quantities of starting material such as are, for example, contained in a forensic sample.

In accordance with the invention this object is satisfied by a method for the determination of the genotype of one or more individuals from a biological sample which contains nucleic acids of different individuals, in partitular a method for determination of the copy number of a predetermined sequence including the steps:
a) making available of at least two aliquots of the biological sample with respectively different concentrations of the biological sample,
b) carrying out at least one amplification reaction in each case with each of the aliquots of the biological sample made available in step a), with the at least one amplification reaction being adapted to amplify one or at least two sequences homologous to another and/or not homologous to one another which are included by at least one of the nucleic acids contained in the biological sample,
c) determination of the obtained number of different amplification products for each of the at least two aliquots with the at least one amplification reaction used in each case in accordance with step b) and comparison of the numbers obtained,
d) when the number determined in step c) of different amplification products obtained for each of the at least two aliquots is the same: making available at least one further aliquot of a biological sample with a respectively different concentration than the aliquots made available in step a) and repeating the steps b) and c) and optionally d) until, in step c) a different number of different amplification products is obtained for at least one aliquot than for the other aliquots,
e) characterization of the amplification products which were obtained with the at least one aliquot for which a different number of different amplification products was obtained in the at least one amplification reaction than for the other aliquots and/or characterization of the amplification products which were obtained for the at least one aliquot, for which a different number of different amplification products was obtained in the at least one amplification reaction than for the other aliquots, and which were also obtained for the other aliquots.

The making available of at least two aliquots of the biological sample with a different concentration of the biological sample in each case in accordance with step a) of the method of the invention can take place in the manner known to every person skilled in the art for this purpose. For example at least two aliquots can for example be taken from the biological sample for the preparation of the at least two aliquots which are subsequently diluted with dilution factors which are different from one another. It is equally well possible to take two aliquots of the sample and to concentrate one sample whereas the other aliquot is either left undiluted or is diluted. Every other method which makes available at least two aliquots in the biological sample with respect to the different concentrations of the biological sample can be used in step a).

In the first case sketched above that at least two aliquots are taken in step a) which are subsequently diluted with a dilution factor different from one another a method of the invention includes for example the following steps:
a) making available an aliquot of a biological sample,
b) carrying out at least one amplification reaction with the aliquot of the biological sample, with the at least one amplification reaction being adapted to amplify one or at least two sequences which are homologous to one another or not homologous to one another and which are included in at least one of the nucleic acids contained in the biological sample,
c) determination of the number of the different amplification products that are obtained for each of the at least one amplification reactions of step b),
d) making available a further aliquot of the biological sample, dilution of the further aliquot of the biological sample and carrying out at least one amplification reaction under the same conditions as in step b) with the diluted sample,
e) determination of the number of different amplification products obtained for each of the at least one amplification reaction of step d),
f) comparison of the number of different amplification products determined in step c) with the number of different amplification products determined in step e),
g) when the number determined in step c) is the same as the number determined in step e): repetition of the steps d) to f) with a higher dilution factor of the biological sample until a smaller number of different amplification products is obtained in step e) than in step c),
h) characterization of the amplification products, which were obtained in the at least one amplification reaction of step b) not however in the at least one amplification reaction of step d) with a dilution factor with which a smaller number of different amplification products was obtained in step e) than in step c) and/or characterization of the amplification products which were obtained both in the at least one amplification reaction of step b) and also in the at least one amplification reaction of step d) with a dilution factor with which a smaller number of different amplification products was obtained in step e) than in step c).

In the sense of the present invention the determination of the genotype of one or more individuals will be understood as the characterization of at least one predetermined sequence of an individual with respect to the presence or absence, copy number or nuclear acid sequence, i.e. in particular the determination of the absolute or relative number of a predetermined sequence, for example of a genome, of a gene or a gene section, and/or the determination of the presence or absence of a predetermined sequence.

Furthermore, the term different individual in the sense of the invention includes not only—in case of humans—different perons but rather in particular also different cell types of a person which differ from another with respect to their genotype. Examples for this are genetic mosaics or chimeras, i.e. cells of a different genotype of a person, which are first formed by mixing or exchange of different genotypes (chimeras) or arise in an individual (genetic mosaic). An example for a genetic mosaic are cancer cells which arise through LOH (“loss of heterozygosity”).

Moreover, the term homogenous sequence in the sense of the present invention designates sequences which have a similarity among one another with respect to their nucleotide sequence of at least 70%, preferably of at least 80%, particularly preferably of at least 90% and quite particularly preferred of at least 95%, whereas non-homologous sequences are those which have a correspondingly lower sequence similarity among one another.

Furthermore, relative quantitative determination of the number of a predetermined sequence in a biological sample in the sense of the present invention signifies the determination whether a biological sample contains less than, as many or more copies of a predetermined sequence than a reference sample and absolute quantitative determination of the number of a predetermined sequence in the sample signifies the determination as to which specific number of copies of the predetermined sequence are contained in the biological sample.

In distinction to the method known from the prior art for the determination of the genotype of an individual from a biological sample, the method of the invention is not only suited for biological samples which contain the DNA of an individual but rather in particular also for biological samples which contain nucleic acids of at least two different individuals. In accordance with the invention it is achieved in that an amplification reaction is first carried out with one aliquot of the undiluted biological sample which is adapted to amplify one or at least two sequences which are homologous to one another and/or not homologous, which are included by at least one of the nucleic acids contained in the biological sample and in that the number of the different amplification products obtained with the at least one amplification reaction is compared with the number of the amplification products obtained of at least one amplification reaction carried out under the same conditions with an aliquot of the biological sample diluted in such a way that fewer amplification products are obtained in the amplification reaction for the diluted aliquot than with that carried out on the undiluted aliquot of the biological samples. The principle of the method of the invention accordingly relates to diluting an aliquot of the biological sample containing nucleic acids of different individuals so long until at least a part of the theoretically possible amplification products is no longer obtained, with the “missing” amplification products as a rule being those from the DNA present in the biological sample in the lowest concentration. In a heterogeneous DNA mixture such as for example a sample containing nucleic acids of different individuals, with the nucleic acids of the single individuals in the biological sample being present in different quantities, the sequences of the nucleic acids of the single individuals are present in a different copy number. Since a PCR exponentially amplifies the individual target sequences, this unequal distribution quantity-wise can be amplified to such an extent that the target sequence, i.e. the sequence to be amplified by the primer used in the amplification reaction of the DNA of an individual which is present in the smallest concentration, is present in relationship to the corresponding target sequence of the higher concentrated DNA of another individual to such a small extent that it can no longer be detected. This is analogous to the effect termed the “allelic drop-out” in which, in a biological sample containing DNA of only one individual one of the different alleles is amplified. The present invention thus relates to the surprising recognition obtained with experiments on single cells that these dropout transitions arise very sharply.

As a result of the above-named principle and of the above-named associations, the individual amplification products can be associated with the single individuals from whom/which nucleic acids is contained in the biological starting sample by the comparison of the number of amplification products obtained with the undiluted aliquot of the biological sample with the number of amplification products obtained with a correspondingly diluted aliquot of the biological sample, with the dilution factor being so high that fewer amplification products are obtained with the diluted sample than with the undiluted sample. Whereas the amplification products obtained both with the undiluted and also with the diluted sample are namely to be associated with the individual from whom/which a larger quantity of the nucleic acid is contained in the biological sample, the amplification products obtained with the non-diluted biological sample, which are no longer obtained with the diluted biological sample can be associated with the individual from whom/which a smaller quantity of DNA is present in the biological sample. By further characterizing the amplification products which are associated in this way with the single individuals of the biological sample a conclusion can be drawn on the genotype of the individually different individuals.

This can be explained in more detail with reference to a conceptual experiment. Small traces of a biological sample are found at a crime scene which contains nucleic acids of two different individuals (which is not first known), namely nucleic acid of the victim and nucleic acid of the perpetrator. In this connection (which is likewise not initially known), the nucleic acid of the victim is present in the biological sample in a larger quantity than that of the perpetrator. A PCR with a primer pair is now carried out which is adapted to amplify precisely one nucleic acid sequence from, for example, the chromosome 18. Since a healthy human has two chromosomes 18 precisely two amplification products are expected for a heterozygous carrier of the chromosome 18. If both the victim and also the perpetrator are each heterozygous with respect to the chromosome 18 then the theoretically maximum possible number of amplification products for the amplification reaction carried out with one aliquot of the above-named biological sample is four amplification products with two amplification products being obtained for the two alleles of the victim and two amplification products for the two alleles of the perpetrator. If one aliquot of the biological sample is now successively diluted then, from a specific dilution factor onwards, with which the concentration of the nucleic acid of the perpetrator falls short of a certain minimum concentration in the diluted biological sample, the case arises that only the amplification products for the DNA of the victim are obtained, no longer, however, the amplification products for the DNA of the perpetrator. For this reason one can associate the amplification products obtained with the so diluted aliquot of the biological sample, which for example have a length of 120 and 130 bp, with the individual from whom a larger quantity of DNA is contained in the biological sample. In contrast those amplification products which were admittedly obtained with the amplification reaction carried out with the undiluted aliquot of the biological sample, not however with the amplification reaction carried out with the diluted aliquot of the biological sample, which for example have a length of 125 and 135 bp, associate them with that individual from whom a smaller quantity of DNA is contained in the biological sample. Through further characterization of the individual amplification products, conclusions can now be drawn regarding the genotype of the two individuals. By way of example the individual amplification products can be compared with the amplification products obtained with a PCR carried out with a reference sample containing cells of the victim, so that a precise association can be made as to which of the two DNA samples originated from the victim. If the reference sample of the victim for example contains two PCR products with a length of 120 and 130 bp, then a conclusion can be drawn from the above-named experiment with a certain probability that DNA of at least two different individuals is present in the biological sample found at the crime scene or with at least one DNA originating from the victim. In addition the experiment permits the conclusion to be drawn that the DNA present in the biological sample with higher concentration is to be associated with the victim and that the DNA which results in two amplification products with the length of 125 and 135 bp is not to be associated with the victim, but rather with the perpetrator or a third party who does not participate in the crime. The amplification products that are obtained can then be intentionally further analyzed for the perpetrator or the non-participating third party in order, for example, to draw a conclusion as to be identity of the perpetrator by comparison with the data stored in a data bank.

A further advantage of the method of the invention lies in the fact that it is possible to dispense with the determination of the absolute fluorescent intensity of PCR products which is essential in the methods known in the prior art. On the contrary, in the method of the invention, only the numbers of the different amplification products which are respectively obtained with the at least one amplification reaction are determined and compared with one another. In this respect fluorescence marked primers need not necessarily be used in the method of the invention. As far as these are nevertheless used for the detection of the number of different amplification products obtained, it is not necessary to determine the absolute fluorescent intensity of the fluorescence marked amplification products that are obtained, but rather it is only necessary to evaluate whether a fluorescence which, if applicable, lies above a defined threshold value (for example factor 10 or 100) at a wavelength corresponding to the fluorescence factor that is used, is present or not. Accordingly the method of the invention can be carried out simply and at favorable cost without costly apparatus for the qualitative detection of fluorescence.

The method of the invention is basically suitable for the determination of the genotype of one or more individuals from a biological sample containing nucleic acids of different individuals, in dependence on specific number of different individuals. Particularly good results are however obtained if the biological sample contains nucleic acid of at least two but less than or equal to 10 different individuals. The biological sample preferably contains nucleic acids of at least two but less than or equal to five different individuals and quite particularly preferred of two or three and most preferred of precisely two different individuals.

The present invention is also not limited with respect to the quantities or the concentration differences of the individual nucleic acids among one another. The concentration difference of the nucleic acids contained in the biological sample among one another from the single individuals amounts to between 1:1,000 and 1:1, particularly preferably between 1:500 and 1:5 and quite especially preferably between 1:100 and 1:10.

As already explained above, the method of the invention is in particular suitable for forensic investigations, for example in connection with the solving of a crime. However, the method of the invention is not restricted to this but can rather be used for every type of biological sample which contains nucleic acids of at least two different individuals.

A further preferred application of the method of the invention is for example the determination of the genotype of one or more individuals from a biological sample containing a mother's blood, i.e. maternal blood and also fetal cells. Fetal cells arise with a frequency of about 1:1,000,000 in maternal blood. Despite the relatively small number of fetal cells in the maternal blood conclusions can be drawn rapidly and simply regarding the genotype of the fetus with the method of the invention. For this purpose an amplification reaction is first carried out with an aliquot of the undiluted sample which is for example adapted to amplify for example 15 different amplification products from the genome of the mother and then a dilution series is prepared with a further aliquot of the biological sample, with the dilution factor between the individual dilution steps amounting for example to 1:2. Thereafter an amplification reaction is carried out with each dilution step exactly under the same conditions as with the undiluted sample and for each amplification reaction the number of the different amplification products that is obtained is determined. Those amplification products which were admittedly obtained with the aliquot of the undiluted biological sample, not however with the diluted aliquots of the biological sample can be associated with the fetus whereas the other amplification products, i.e. those which were obtained both with the undiluted biological sample and also with the diluted biological sample can be associated with the mother. By characterization of the amplification products it can, for example be determined whether the fetus suffers trisomy 21 or is healthy in this respect. The particular advantage of the method of the invention lies in the fact that this characterization can take place from a biological sample which contains both maternal blood and also fetal cells, without the fetal cells having to be isolated from the maternal blood as is necessary in the prior art.

Furthermore, the method of the invention can also be used to characterize cancer cells which have arisen by LOH (“lost of heterozygosity”) from a mixed sample, for example to typify it. In this case, the biological sample for example contains a mixture of healthy cells and cancer cells which have arisen by LOH.

The method of the invention is not restricted with regard to the nature of the at least one amplification reaction, on the contrary all conceivable types of amplification reaction can be used with which sequence variants can be detected. Nevertheless, it has proved advantageous to carry out a PCR as the at least one amplification reaction because a PCR can be carried out simply, comparatively quickly and also with small technical costs and complexity and desired nucleic acid sequences from the biological sample can be amplified by the choice of suitable primer pairs.

Since the genotype of one or more individuals is to be determined with the method of the invention from a biological sample containing nucleic acids of different individuals. it is proposed, as a further development of the concept of the invention, to adapt the at least one amplification reaction to amplify one or at least two sequences which are homologous and/or non-homologous to one another from the non-coded DNA range. In known manner the non-coded DNA range is substantially more polymorphic than the coded DNA range so that by amplification of sequences from the non-coded DNA range sequences specific to individuals can be amplified with a relative large probability. This is advantageous both with forensic mixed samples and also in a characterization of the genotype of fetal cells from maternal blood containing fetal cells.

Furthermore, it has proved advantageous to adapt the at least one amplification reaction to amplify one or at least two highly polymorphic sequences which are homologous to one another and/or non-homologous. Good results are obtained, in particular in cases in which the at least one amplification reaction is adapted to amplify one sequence or at least sequences homologous and/or non-homologous to one another which are selected from the group consisting of STR sequences, VNTR sequences, SNP sequences and desired combinations hereof. STR sequences, i.e. short tandem repeat sequences, are highly polymorphous sequences which consist of only two to four bp long repetition units which have a high variability between the single individuals. In distinction to this VNTR sequences, i.e. variable number of tandem repeat sequences, consist of repetitive DNA sections of about 15 to 30 bp length, the total length of which are determined by the number of repetitions of this basic unit. VNTR sequences are as a rule also highly polymorphic, i.e. the number of the individual repetition units is very strongly distinguished between the different individuals. SNP's (single nucleotide polymorphism) are the simplest polymorphisms in which the homologous sequences are only distinguished by a base in each case. These sequences are also excellently suited for the carrying out of the method of the invention since these are very strongly distinguished between the single individuals. Apart from this all other highly polymorphic sequences are however also suitable as a marker for the method of the invention.

Furthermore, it is preferred that the at least one amplification reaction is adapted to amplify one sequence or at least two sequences which are homologous to one another and/or non-homologous which only arise once per allele respectively in the genome of the donor. Thus, in the characterization of the amplification products conclusions can be drawn on the individual alleles on an individual so that, for example, the number of individual alleles of an individual in the biological sample including the nucleic acid of different individuals can be determined.

The at least one amplification reaction is preferably adapted to amplify between 1 and 100, preferably between 2 and 20 and particularly preferably between 5 and 15 sequences which are homologous to one another and/or non-homologous. In this way sufficient different amplification products are obtained in order to obtain targeted individual specific results in the characterization of the amplification products. On the other hand, the experimental cost and complexity is not yet too large.

In accordance with a preferred embodiment of the present invention, a PCR adapted for the amplification of at least two sequences which are homologous to one another and/or not homologous is carried out in step b) and/or step d) of the method of the invention. In the PCR a number of primer pairs is used corresponding to the number of the at least two sequences which are homologous to one another and/or not non-homologous and which are adapted to amplify the at least two sequences which are homologous to one another and/or not homologous. An advantage of this embodiment lies in the fact that in each case only one PCR is necessary both with the amplification reaction carried out with the undiluted aliquot of the biological sample and also with the amplification reaction carried out with the dilution stage or stages of the aliquot of the biological sample, so that the method can be carried out quickly and without a large pipetting effort. An example for suitable method guidance is a multiplex PCR, however, any other amplification reaction can be used in which the one sequence to be amplified, or the at least two sequences which are homologous to one another and/or not homologous to be amplified, can be simultaneously amplified in one reaction.

In accordance with a further preferred embodiment of the present invention a PCR adapted for the amplification of at least two sequences which are homologous to one another and/or not homologous is carried out in step b) and/or step d) and in step a) a number of aliquots of the biological sample is made available which corresponds to the number of the at least two sequences which are homologous to one another and/or not homologous, with each aliquot containing the some quantity of biological material and in step b) and/or step d) a PCR is carried out with each of the aliquots in which a primer pair is used in each case, with the primer pairs used in the different PCRs being adapted to amplify the at least two sequences which are homologous to one another and/or not homologous. An advantage of this way of carrying out the method lies in the fact that the individual amplifications cannot mutually influence one another, possibly negatively.

In the above-named embodiment in particular, but also in cases in which a large number of dilution stages is necessary in order to obtain a smaller number of amplification products with the diluted sample than with the undiluted sample, it can be necessary to amplify the biological sample, for example with a non-specific PCR, prior to carrying out the method step a), i.e. prior to making available an aliquot of the biological sample, in order to have sufficient starting material in order to be able to make available the necessary number of aliquots of the biological sample.

Apart from the two above-named embodiments of the present invention, mixed forms of both embodiments are also conceivable, for example one in which a part of the at least two sequences to be amplified which are homologous to one another and/or not homologous are amplified in a PCR using at least two primer pairs and the other part of the at least two sequences to be amplified which are homologous to one another and/or not homologous are each amplified in PCRs separate from this with only one primer pair being used in each case in these PCRs.

In order to be able to determine the number of different amplification products that are obtained, the presence or absence of amplification products must first be determined. For the determination of the presence or absence of amplification products, all methods known to the person skilled in the art for this purpose can be used; simply by way of example gel electrophoresis, familiar hybridization techniques, for example those of a DNA array are to be named. In this connection it can be expedient, in dependence on the detection method that is used, to define threshold values above which the presence of a PCR product is assumed and below which the absence of a PCR product is assumed.

In addition to the determination of the presence or absence of an amplification product, a second preferably physically and/or chemically measurable parameter must be determined for the determination of the number of different amplification products that are obtained in order to be able to distinguish the individual amplification products from one another. In this connection the nature of the second parameter which distinguishes the individual PCR products from one another depends essentially on the type of the one sequence or at least two sequences which are to be amplified which are homologous to one another and/or not homologous. If for example the PCR primers are so selected in the at least one amplification reaction that STR sections and/or VNTR sections are amplified as sequences which are homologous to one another and/or non-homologous, then the length of the individual PCR products is preferably selected as the second parameter or as the second distinguishing feature of the individual PCR products, so that the determination of the number of the different amplification products that is obtained includes the examination for the presence or absence of PCR products and also the determination of the length of the individual PCR products, with the number of the different amplification products obtained corresponding to the number of the amplification products that are obtained with different length. A suitable method for this is for example capillary electrophoresis.

If, in contrast, PCR primers are used in at least one amplification reaction which are adapted to amplify one SNP sequence or at least two SNP sequences which are homologous to one another and/or not homologous, then the second distinguishing feature, i.e. of the second parameter, is preferably the determination of the different sequence, which with SNP sections is normally restricted to one nucleotide. For this all methods known for the person skilled in the art for this purpose can be used with DNA sequencing or known hybridization methods being named simply by way of example.

In the context of the present invention it has proved advantageous to set the parameters in the at least one amplification reaction in accordance with step b) and/or in accordance with step d) such that the relative frequency for a positive amplification reaction for the one sequence or for each of the at least two sequences which are homologous to one another and/or not homologous is in each case at least substantially of the same size. Thus, the sequence to be amplified of the nucleic acids of the different individuals contained in the biological sample is amplified with the same effectivity, providing it is present in an equally large quantity, so that a conclusion can reliably be drawn, after the loss of an amplification product from a certain dilution stage onwards, that the loss of the amplification product is to be attributed to the fact that the DNA of the corresponding individual is present in the biological sample in a correspondingly smaller quantity than that of the other individual and that the loss is not simply attributed to the fact that the effectiveness of the amplification reaction for this amplification product was lower, even with the same DNA quantity than that for another amplification product. Accordingly, it is preferred, in accordance with the invention, to set the binding affinity of the individual PCR primers to their primer binding sets and also the other parameters of the PCR, in particular the number of cycles and the temperature control such that the relative frequency for a positive amplification reaction of the at least one amplification reaction amounts for each of the sequences to be amplified which are homologous to one another and/or not homologous amounts to between 0.2 and less than 1, preferably to between 0.4 and 0.6 and also particularly preferably amounts to 0.5. If the relative frequency for a positive amplification reaction for the sequences to be amplified which are homologous to one another and/or not homologous were 1, a loss of the amplification product for the nucleic acid present in the biological sample in a smaller DNA concentration would first be observed from a relatively high dilution stage onwards. Accordingly, it is advantageous to set the relative frequency for a positive amplification of the at least one amplification reaction to a value of less than 1. In order, on the other hand, to prevent a loss of the PCR products being observed even with the undiluted sample a relative frequency for a positive amplification reaction should on the other hand not be too low.

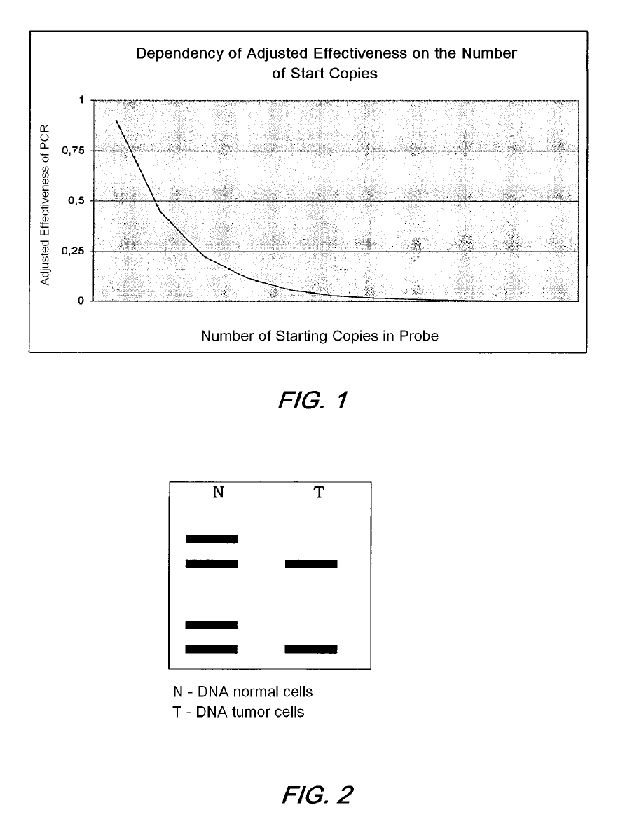

However, account must be taken of the fact that the above-named values for the relative frequency to be set for a positive amplification reaction of the at least one amplification reaction for each of the sequences to be amplified (termed in the following also effectivity) are not fixed values but rather depend in particular on the number of the starting copies used in the PCR. The larger the number of starting copies, the smaller the effectiveness of the at least one amplification reaction should be set in order to achieve a loss of the amplification products for the nucleic acid present in a low DNA concentration in the biological sample from a relatively small dilution stage onwards. This dependence of the effectivity to be set on the number of starting copies, i.e. on the number of the cells that is used or on the number of the copies that is used is shown in FIG. 1.

In a further development of the concept of the invention it is proposed to carry out an amplification reaction under the same conditions with a control sample parallel to the at least one amplification reaction in accordance with step b) and/or step d). In this connection the control sample preferably leads to a known number of different amplification products. In this way it can be determined in simple manner whether the at least one amplification reaction in accordance with step b) and/or step d) has taken place in an orderly manner, or, whether, this has not taken place or taken place inadequately, possibly due to a defect of the thermocycler.

The dilution factor to be selected for the aliquot of the biological sample depends in particular on the concentration of the nucleic acid in the biological sample and can be easily determined in the context of normal specialist investigations by the person skilled in the art. It has however basically proved advantageous to dilute the aliquot in the biological sample in a ratio between 1:1 and 1:1,000, preferably between 1:1 and 1:100, particularly preferably between 1:1 and 1:10 and especially preferably between 1:1 and 1:2.

For the characterization of the amplification products all techniques known to the person skilled in the art can be used which permit a conclusion to be drawn concerning the genotype of the corresponding individual. By way of example the characterization of the amplification products can include the determination of the relative number of one or more alleles of a predetermined sequence.

For the determination of relative number of alleles of a predetermined sequence, for example of a chromosome, a gene or a gene section, every method known to the person skilled in the art for this purpose can be used. By way of example this can take place in that at least one amplification reaction is carried out under the same conditions as in step b) of the method of the invention with a reference sample and a number of the different amplification products obtained with this at least one amplification reaction with the reference sample is compared with the number of the amplification products obtained only for a part of the aliquot. Preferably the reference sample has a known genotype; for this purpose it is for example sufficient to know whether the individual from whom the reference sample was taken is a healthy or sick individual with respect to the predetermined sequence on which the allele to be determined is present. The copy number of the predetermined sequence of the reference sample can however equally well also be known. Moreover, it is preferred that the same size of DNA quantity is used in the reference sample in the at least one amplification reaction as in the at least one amplification reaction in accordance with step b) of the method of the invention. In order to ensure this it can for example be necessary to determine the DNA concentration in the biological sample—optionally after a non-specified PCR or the augmentation of the material. By comparing the number of the different amplification products obtained for only one part of the aliquots, i.e. the amplification products for the individual from whom the larger quantity of DNA is contained in the biological sample, with the corresponding number of different amplification products obtained for a reference sample with the same amplification reaction, the relative number of the alleles of a predetermined sequence can be determined providing corresponding primer pairs are used in the at least one amplification reaction which are adapted to amplify the alleles included by the predetermined sequence.

As an alternative to the above-named embodiment it is however also possible, for the characterization of the amplification products, to carry out at least one amplification reaction under the same conditions as in step b) with the reference sample and to compare the number of the different amplification products obtained with this at least one amplification reaction with the number of the different amplification products obtained for all aliquots. In distinction to the previously named embodiment the relative number of the alleles of a predetermined sequence in the individual can be determined from the smaller DNA amount contained in the biological sample.

In a further development of the concept of the invention it is proposed, for the characterization of the amplification products, to compare the number of the different amplification products obtained only for a part of the aliquots and/or the number of the different amplification products determined for all aliquots of the biological sample with at least one frequency distribution, with the frequency distribution for example being obtained by separate in each case multiple carrying out of the same at least one amplification reaction as used in step b) and under the same reaction conditions, with the same quantity of starting material being used in the at least one amplification reaction as was/is is used in step a) with at least two different reference samples, with the at least two different reference samples each having a known copy number of the predetermined sequence different from one another and also with subsequent determination of the number of different amplification products that was/is obtained per reference sample. In this manner a particularly reliable determination of the relative or indeed absolute number of the alleles of a predetermined sequence in each of the individuals is possible from whom/which nucleic acids are contained in the biological sample.

Furthermore it is possible, for the characterization of the amplification products, to sequence the amplification products that are obtained or to subject them to a hybridization process with suitable probes, for example to obtain conclusions regarding the sequence of a gene or of a gene section.

Finally, for the characterization of the amplification products a multiple determination of the PCR can also be carried out with one or more dilution stages of the biological sample in order to conclude, for example, the relative or absolute number of the predetermined sequence in the biological sample from the comparison of the average value of the numbers of different amplification products that are obtained in the individual determinations. It is equally well possible to carry out a multiple determination of the PCR with one or more dilution stages of the biological sample and to compare the average value of the number of an obtained specific amplification product, for example of an allele-specific amplification product, with the average value of the number of another specific amplification product, for example of an allele-specific amplification product. By way of example, in each case, a five-times determination of a PCR can be carried out for the dilution stages 1:5 and 1:10 of a biological sample, with the primer pairs used in the PCR being adapted to amplify at least one allele-specific sequence. If, for example, two amplification products are obtained with the PCR for the allele-specific primer pair for an individual and if the two amplification products appear equally frequently in the individual dilution stages, for example on an average by in each case of 0.5-times with the dilution stage 1:5 and 0 times with the dilution stage of 1:10 then a conclusion can be drawn of the existence of a biallelic disomy. If, however, two allele-specific amplification products are obtained in the individual dilution stages of which one occurs twice as frequently as the other, for example amplification product 1 on average 0.9 times in the dilution stage. 1:5 and an amplification product 2 on average 0.5 times in the dilution stage 1:10 or if one first drops out at a higher dilution factor than the other, for example amplification product 1 is no longer obtained for the first time in the dilution stage 1:10, whereas the amplification product 2 is already no longer obtained in the dilution stage 1:5, then this already indicates a biallelic trisomy. The number of multiple determinations preferably amounts to between 2 and 1,000, particularly preferably to between 3 and 100, quite especially preferably between 4 and 15 and most preferably to between 5 and 10.

A further subject of the present invention is a kit for the determination of the genotype of one or more individuals from a biological sample which contains nucleic acids from different individuals, in particular for carrying out the above-named method including:
a) at least one primer pair which is adapted to amplify, in at least one PCR, one sequence or at least two sequences homologous to one another and/or not homologous, which are included by at least one of the nucleic acids contained in the biological sample,
b₁) a reference sample with a known genotype and preferably with a copy number known with respect to a predetermined sequence and/or
b₂) the result of at least one amplification reaction carried out with a reference sample under the same conditions as described in the protocol in accordance with d), wherein the reaction conditions were so selected that the at least one amplification product arose with a probability between 20% and less than 100% and/or
b₃) at least one frequency distribution which was obtained by separate in each case multiple carrying out of the same at least one amplification reaction with at least two different reference samples carried out under the same reaction conditions as prescribed in the protocol d), with the at least two different reference samples each having a known copy number of a predetermined sequence different from one another, and also subsequent determination of the number of different amplification products obtained per reference sample, and
c) if required PCR puffer and
d) a protocol for carrying out the at least one PCR in a) and, if required, particulars of the dilutions to be effected with the at least two aliquots with different concentration.

In accordance with the preferred embodiment of the present invention the kit includes a reference sample b₁) with a known genotype and preferably with a known copy number with respect to a predetermined sequence. By carrying out the same at least one amplification reaction, with the reference sample of the known genotype as with the individual aliquots of the biological sample, a conclusion can be drawn in the characterization of the amplification products in the context of the method of the invention by comparison of the number of amplification products obtained for one of the aliquots of the biological sample with the number of the different amplification products obtained for the reference sample in the at least one amplification reaction regarding the relative copy number of the predetermined sequence in the nucleic acid of an individual contained in the biological sample. When at least two different aliquots from the reference sample of different concentrations are respectively multiply subjected to at least one amplification reaction under the same conditions as the aliquots of the biological sample to be investigated, then a conclusion can indeed be drawn regarding the absolute copy number of the predetermined sequence in the nucleic acid of an individual contained in the biological sample.

Instead of a reference sample or, even though less preferred additionally to a reference sample b₁) the kit in accordance with the invention can include the result b₂) of at least one amplification reaction with a reference sample carried out under the same as prescribed in the protocol in accordance with d), with the reaction conditions being so selected that the at least one amplification product was formed with a probability between 20% and less than 100% and/or include at least one frequency distribution b₃) which was obtained by separate in each case multiple carrying out of the same at least one amplification reaction with at least two different reference samples and under the same reaction conditions as prescribed in the protocol d), with the at least two different reference samples each having a known copy number of a predetermined sequence different from one another and also with subsequent determination of the number of different amplification products obtained per reference sample. Whereas a comparison of the result in accordance with b₂) with the number of different amplification products obtained in the at least one amplification reaction carried out with an aliquot of the biological sample in accordance with the protocol d) permits the determination of the relative copy number of a predetermined sequence in the nucleic acid of an individual contained in the biological sample, a corresponding comparison with the frequency . distribution in accordance with b₃) enables the determination of the absolute copy number of a predetermined sequence in the nucleic acid of an individual contained in the biological sample.

In accordance with the preferred embodiment of the kit in accordance with the present invention the at least one primer pair is adapted to amplify in the at least one PCR one sequence or at least two sequences which are homologous to one another and/or not homologous from the non-coded DNA range, preferably highly polymorphic sequences which are homologous to one another and/or not homologous which are particularly preferably selected from the group consisting of STR sequences, VNTR sequences, SNP sequences and desired combinations hereof.

In a further development of the concept of the invention it is proposed that the at least one primer pair in accordance with a) and/or the protocol in accordance with d) of the kit in accordance with the invention be adapted to amplify in the at least one PCR between 1 and 100, preferably between 2 and 20 and particularly between 5 and 15 sequences which are homologous to one another and/or not homologous.

In the following the present invention will be explained with reference to examples which explain it but do not restrict it:

### EXAMPLE 1

In the present example the genotype of fetal cells present in maternal blood is to be determined.

Fetal cells are present in maternal blood with the frequency of about 1:1,000,000. Fetal cells can be enriched in maternal blood by means of different methods. For example, magnetic beads with specific antibodies for fetal cells can be used for this purpose or cell sorters which recognize the fetal cells with respect to membrane protein and separate them. In this manner the fetal cells in the maternal blood can be enriched up to a ratio of 1:1,000.

In the following a determination should be made with respect to a so concentrated sample whether the fetus is affected by trisomy 21 or not. In this example it is known from a cell sorter experiment that approximately 10,000 cells are present in the sample. Furthermore, it is known from reference experiments that for the STR system used here (with the same protocol as in the reference experiments) a dropout, i.e. non-amplification of the amplification products included by the primer pairs that are used, arises when, with two copies of the chromosome 21 per cell, less than 10 cells are used in the PCR or, with 3 copies of the chromosome 21 per cell, less than 7 cells are used in the PCR.

First of all an aliquot of a concentrated mixed sample containing fetal cells and maternal blood in a ratio of 1:1,000 was subjected to a PCR with one primer pair, with the primer pair having been adapted to amplify in each case one STR sequence of the chromosome 21. For a healthy homozygotic individual with respect to the chromosome 21 one amplification product is expected, for a healthy heterozygote individual with respect to chromosome 21 two amplification products are expected, for an individual with monoallelic trisomy one amplification product is expected, for an individual with biallelic trisomy two amplification products are expected and for an individual with triallelic trisomy three amplification products are expected. Moreover, with respect to a further aliquot of the biological sample, a dilution series was pipetted and with an aliquot of each dilution stage the same PCR was carried out as with an aliquot of the non-diluted sample. The following results were obtained:

| Amplificat length | Dilution factor | | | | | | | |
|---|---|---|---|---|---|---|---|---|
| (bp) | 0 | 1:2 | 1:5 | 1:7 | 1:10 | 1:100 | 1:500 | 1:1,000 |
| 132 | pos | pos | pos | pos | pos | Pos | pos | pos |
| 136 | pos | pos | neg | neg | neg | Neg | neg | neg |
| 144 | pos | pos | pos | pos | pos | Pos | pos | neg |
| 156 | pos | pos | pos | neg | neg | Neg | neg | neg |

pos = positive (amplification product obtained)
neg = negative (no amplification product obtained)

As is evident from the table the amplification products with a length of 132 bp and 144 bp were obtained up to a dilution stage of 1:1,000 and 1:500 respectively, whereas the two other amplification products with a length of 136 bp and 156 bp were already lost from a dilution stage of 1:5 and 1:7 on respectively. From this it follows that the alleles with the length 136 bp and 156 bp are to be associated with the fetus since they dropout with substantially lower dilutions than the two other alleles with the length 132 bp and 144 bp. This result can be verified by way of a comparison with maternal cells from tissue.

The above result does not however permit a reliable conclusion whether the alleles with the lengths 136 bp and 156 bp are present in one copy or in two copies. For this purpose five experiments were carried out in parallel with aliquots of the biological sample of the dilution stage 1:5, in each case with the same amplification reaction as the named amplification reaction. In principle this corresponds to a five-times multiple determination. In this connection the following result was obtained:

| Amplificat length (bp) | Exp. 1 | Exp. 2 | Exp. 3 | Exp. 4 | Exp. 5 |
|---|---|---|---|---|---|
| 136 | pos | pos | pos | neg | pos |
| 156 | neg | pos | pos | neg | neg |

Exp.: experiment

Thereafter the same experiment was in each case carried out five times with an aliquot of the dilution stage 1:7, the following result was obtained:

| Amplificat length (bp) | Exp. 1 | Exp. 2 | Exp. 3 | Exp. 4 | Exp. 5 |
|---|---|---|---|---|---|
| 136 | pos | neg | neg | neg | Pos |
| 156 | neg | neg | neg | neg | neg |

It is evident from the two above tables that the allele with the length of 156 bp shows a significantly higher probability of dropout, i.e. lack of the expected amplification product in a given dilution stage than the allele with a length of 136 bp. From this it can be concluded with a certain probability that the allele with the length 136 bp is present with, in each case, two copies per cell and the allele with the length 156 bp is present in one copy per cell. If the two alleles were present with the same copy number per cell than the dropouts at the same dilution should also be equally frequent.

Consequently, the above experiments allow pronouncements to be made concerning the relative number of alleles from an individual from a biological sample which contains nucleic acids from two different individuals. In this specific case a biallelic trisomy 21 is diagnosed for the fetus. As the person skilled in the art recognizes, the reliability of the pronouncements that are made concerning the above tests are increased if one were to use more than two primer pairs in the amplification reaction.

### EXAMPLE 2

The same procedure took place as described in example 1, however, a different biological sample of maternal blood containing fetal cells was used in the PCR. In this connection the following result was obtained:

| Amplificat | | | | | | | | |
|---|---|---|---|---|---|---|---|---|
| length | Dilution factor | | | | | | | |
| (bp) | 0 | 1:2 | 1:5 | 1:7 | 1:10 | 1:100 | 1:500 | 1:1,000 |
| 132 | pos | Pos | pos | pos | pos | pos | pos | pos |
| 136 | pos | Pos | neg | neg | neg | neg | neg | neg |
| 144 | pos | Pos | pos | pos | pos | pos | pos | neg |
| 156 | pos | Pos | pos | neg | neg | neg | neg | neg |
| 160 | pos | Pos | neg | neg | neg | neg | neg | Neg |

pos = positive (amplification product obtained)
neg = negative (no amplification product obtained)

As can be seen from the above table, the amplification products with the length of 136 bp, 156 bp and 160 bp drop out at a lower dilution stage than the corresponding amplification products with the length of 132 bp and 144 bp. From this it follows that the three first-named amplification products are to be associated with the fetal cells present in a smaller quantity in a biological sample, whereas the two last-named amplification products are to be associated with the mother. Since three amplification products for the fetus are obtained for the undiluted sample and in contrast only two amplification products were obtained for the mother, one can make the statement with a large probability that the fetus suffers under triallelic trisomy of the chromosome 21.

### EXAMPLE 3

In the following a forensic mixed sample, namely a biological sample containing nucleic acids of different individuals found at a crime scene for a violent crime is to be characterized. For this an amplification reaction was carried out as in example 1 with different dilution stages of the biological sample and the following result was obtained:

| Amplificat | | | | | | | | |
|---|---|---|---|---|---|---|---|---|
| length | Dilution factor | | | | | | | |
| (bp) | 0 | 1:2 | 1:5 | 1:7 | 1:10 | 1:100 | 1:500 | 1:1,000 |
| 132 | pos | Pos | pos | pos | pos | pos | neg | neg |
| 136 | pos | Pos | neg | neg | neg | neg | neg | neg |
| 144 | pos | Pos | pos | pos | pos | pos | pos | neg |
| 156 | pos | Pos | pos | neg | neg | neg | neg | neg |

pos = positive (amplification product obtained)
neg = negative (no amplification product obtained)

It is evident from the table that a total of four amplification products were obtained of which two, namely the amplification products with the length 132 bp and 144 bp first drop out at a high dilution stage (1:500 and 1:1,000 respectively) whereas the two other amplification products, namely those with a length of 136 bp and 156 bp already fall out with a lower dilution stage of 1:5 and 1:7 respectively. From this it follows that the biological sample contains nucleic acids of two different individuals, with the alleles with the length 132 bp and 144 bp being associated with the individual 1 whereas the alleles with the length 136 bp and 156 bp are associated with the individual 2. This conclusion is obvious because for these combinations the dropouts occur at similar dilution stages. It is thus very improbable that nucleic acid of a third individual is contained in the biological sample because then two individuals would only be present with one allele in the sample. This is, however, very improbable. The nucleic acids of the two individuals can now be characterized more precisely as described previously.

As the above examples show, both the relative frequency of at least one predetermined sequence can be determined and also an identity determination carried out, i.e. the determination of the copy number of individual alleles, via a dilution series and carrying out corresponding amplification reactions with the undiluted biological sample and individual dilution stages of the biological sample. The difference for the identity determination preferably amounts to about a factor 10. However, even smaller differences are sufficient when, for example, the copy number of alleles of an individual from a mixed trace is to be determined. However, then a certain redundancy is necessary, such as by multiplex PCR or multiple determinations of a PCR with corresponding aliquots in order to increase the statistical reliability.

### EXAMPLE 4

A determination should be made whether a biological sample which can also contain also fetal cells in addition to maternal blood can, for example in the case of a pregnant woman, contain such fetal cells and, if these are present, what the ratio of maternal cells to fetal cells is.

The determination of the relative cell number of fetal cells in maternal blood is not possible from the methods known from the prior art and based on PCR or, if at all possible, is only poorly possible since fetal cells only arise in maternal blood in a frequency of 1 to 1 million cells. In this respect, in the methods known from the prior art more than 1 μg of maternal blood must be used in the PCR in order to obtain an evaluatable result at all. However, a PCR with such high starting quantity of DNA does not run ideally so that only an imprecise result is obtained. In order to circumvent this, it has already been proposed to enrich fetal cells by FACS. However, the ratios are significantly shifted by the enrichment.

Since maternal cells are present in excess in the biological sample in addition to the fetal cells and since both cell types respectively have a diploid chromosome set, there are a maximum of 4 different alleles present in the biological sample for each gene. The hypothesis will always be that the maternal cells are present in excess. In the PCR an allele-specific primer pair was now used for a specific genome section and the following results were obtained.

| | Dilution stage | | | | | | |
|---|---|---|---|---|---|---|---|
| PCR 1 | 1:1 | 1:2 | 1:4 | 1:8 | 1:16 | 1:32 | 1:64 |
| Allele I-1 | pos | pos | pos | neg | neg | neg | neg |
| Allele I-2 | pos | pos | pos | pos | neg | neg | neg |
| Allele II-1 | pos | pos | pos | pos | pos | pos | neg |
| Allele II-2 | pos | pos | pos | pos | pos | neg | neg |

pos: positive PCR reaction
neg: negative PCR reaction
Allele I-1: Allele 1 from cell type I (fetal cells)
Allele II-2: Allele 2 from cell type II (maternal cells)

From the comparison with the number of positive PCRs per dilution stage it can be seen that there are now clearly more maternal cells than fetal cells contained in the biological sample, since during the amplification of the fetal cells DNA copies dropouts occur earlier than with the maternal cells. Thus, first dropouts for the fetal cells are already to be observed with a dilution stage of 1:8, whereas in a dilution stage of 1:16 no amplification products are any longer obtained for the fetal cells. In contrast the drop-outs for the maternal cells first start at a dilution stage of 1:32 before no amplification products are any longer obtained for the maternal cells also from a dilution stage of 1:64 onwards.

The relative statement with respect to both samples from the onset of the dropout (dilution stage 1:64 for the maternal cells in comparison to the dilution stage 1:16 for the fetal cells) which were present in mixed form from the outset is now that the maternal cells are present in approximately 4-times the quantity of the fetal cells.

The fetal cells and/or the maternal cells can now be further characterized as described previously.

### EXAMPLE 5

A determination should be made whether a biological sample of a person which contains cancer cells which have arisen by LOH in addition to healthy cells.

For this purpose an amplification reaction was carried out with different dilution stages of the biological sample with a primer pair amplifying the gene section D85522 and the following result was obtained:

| | Dilution stage | | | | | | |
|---|---|---|---|---|---|---|---|
| PCR 1 | 1:1 | 1:2 | 1:4 | 1:8 | 1:16 | 1:32 | 1:64 |
| Allele I-1 | pos | pos | pos | neg | neg | neg | neg |
| Allele I-2 | pos | pos | pos | pos | neg | neg | neg |

pos: positive PCR reaction
neg: negative PCR reaction

From the table it can be seen that the allele I-2 was contained in all dilution stages, whereas the allele I-1 was only amplified up to a dilution stage 1:16. This shows that more copies of the allele I-2 than of allele I-1 are present in the biological sample. From this a conclusion can be drawn with a certain probability that the biological sample is a mixed sample which contains both healthy heterozygotic cells with respect to the allele I and also cancer cells which have arisen by LOH which are only homozygotic with respect to the allele I.

This result can be validated in that a plurality of aliquots of the biological sample are diluted until these each only contain one cell and subsequently the DNA of each aliquot is non-specifically amplified before the DNA of each aliquot is separated by gel electrophoresis and visualized by Southern-Blot with a probe specific for the named gene section. The result after applying two aliquots is reproduced in FIG. 2.

As can be taken from FIG. 2 the biological sample contains two different cell types of which one contains two alleles of the D85522-gene (the two lower bands in the trace “N”), whereas the other cell type only contains one of the two alleles of the D85522-gene (the lower band in the trace “T”). The bands shown in the upper half of the gel are “confirmation bands” which arise through alternatively folded sequences of each allele.

1. A method for the determination of the genotype of one or more individuals from a biological sample which contains nucleic acids of different individuals, in particular a method for the determination of the copy number of a predetermined sequence, including the steps of:
a) making available at least two aliquots of the biological sample with respectively different concentrations of the biological sample,
b) carrying out at least one amplification reaction in each case with each of the aliquots of the biological sample made available in step a), with the at least one amplification reaction being adapted to amplify one sequence or at least two sequences homologous to another and/or not homologous which are included by at least one of the nucleic acids contained in the biological sample,
c) determination of the obtained number of different amplification products for each of the at least two aliquots with the at least one amplification reaction used in each case in accordance with step b) and comparison of the numbers obtained,
d) when the number determined in step c) of different amplification products obtained for each of the at least two aliquots is the same: making available at least one further aliquot of a biological sample with a respectively different concentration than the aliquots made available in step a) and repeating the steps b) and c) and optionally d) until, in step c) a different number of different amplification products is obtained for at least one aliquot than for the other aliquots,
e) characterization of the amplification products which were obtained with the at least one aliquot for which a different number of different amplification products was obtained in the at least one amplification reaction than for the other aliquots and/or characterization of the amplification products which were obtained for the at least one aliquot, for which a different number of different amplification products was obtained in the at least one amplification reaction than for the other aliquots, and which were also obtained for the other aliquots.

2. A method for the determination of the genotype of one or more individuals from a biological sample which contains nucleic acids of different individuals, in particular a method for determining the copy number of a predetermined sequence, including the steps of:
a) making available an aliquot of the biological sample,
b) carrying out at least one amplification reaction with the aliquot of the biological sample, with the at least one amplification reaction being adapted to amplify one or at least two sequences which are homologous to one another and/or not homologous and which are included in at least one of the nucleic acids contained in the biological sample,
c) determination of the number of the different amplification products that are obtained for each of the at least one amplification reactions of step b),
d) making available a further aliquot of the biological sample, dilution of the further aliquot of the biological sample and carrying out at least one amplification reaction under the same conditions as in step b) with the diluted sample,
e) determination of the number of different amplification products obtained for each of the at least one amplification reaction of step d),
f) comparison of the number of different amplification products determined in step c) with the number of different amplification products determined in step e),
g) when the number determined in step c) is the same as the number determined in step e): repetition of the steps d) to f) with a higher dilution factor of the biological sample until a smaller number of different amplification products is obtained in step e) than in step c),
h) characterization of the amplification products, which were obtained in the at least one amplification reaction of step b) not however in the at least one amplification reaction of step d) with a dilution factor with which a smaller number of different amplification products was obtained in step e) than in step c) and/or characterization of the amplification products which were obtained both in the at least one amplification reaction of step b) and also in the at least one amplification reaction of step d) with a dilution factor with which a smaller number of different amplification products was obtained in step e) than in step c).

3. A method in accordance with claim 1 or claim 2, characterized in that the biological sample contains nucleic acids of at least two different individuals, preferably of at least two but less than or equal to 10, particularly preferably of at least two but less or equal to 5, quite especially preferably from two or three and most preferably from precisely two different individuals.

4. A method in accordance with any one of the preceding claims, characterized in that the concentration difference of the nucleic acids contained in the biological sample from the single individuals amounts to between 1:1000 and 1:1, preferably to between 1:500 and 1:5 and particularly preferably to between 1:100 and 1:10.

5. A method in accordance with any one of the preceding claims, characterized in that a forensic sample is used as the biological sample.

6. A method in accordance with any one of the claims 1 to 4, characterized in that the biological sample includes maternal blood containing fetal cells and preferably consists of maternal blood containing fetal cells.

7. A method in accordance with any one of the claims 1 to 4, characterized in that the biological sample is a mixture of healthy cells and cancer cells arising through LOH.

8. A method in accordance with any one of the preceding claims, characterized in that the at least one amplification reaction is a PCR reaction.

9. A method in accordance with any one of the preceding claims, characterized in that the at least one amplification reaction is adapted to amplify one sequence or at least two sequences which are homologous to one another and/or not homologous from the coded DNA range.

10. A method in accordance with any one of the preceding claims, characterized in that the at least one amplification reaction is adapted to amplify one highly polymorphous sequence or at least two highly polymorphous sequences which are homologous to one another and/or are not homologous to one another.

11. A method in accordance with any one of the preceding claims, characterized in that the at least one amplification reaction is adapted to amplify one sequence or at least two sequences which are homologous to one another and/or not homologous which are selected from the group consisting of STR sequences, VNTR sequences, SNP sequences and any desired combinations hereof.

12. A method in accordance with any one of the preceding claims, characterized in that the at least one amplification reaction is adapted to amplify one or at least two sequences which are homologous to one another and/or not homologous to one another which only are present once per allele in the genome of the donor.

13. A method in accordance with any one of the preceding claims, characterized in that the at least one amplification reaction is adapted to amplify between 1 and 100, preferably between 2 and 20 and particularly preferably between 5 and 15 sequences which are homologous to one another and/or not homologous to one another.

14. A method in accordance with any one of the preceding claims, characterized in that in step b) and/or in step d) a PCR is carried out which is adapted for the amplification of at least two sequences which are homologous to one another and/or not homologous with a number of primer pairs being used in the PCR corresponding to the number of at least two sequences which are homologous to one another and/or not homologous and which are adapted to amplify the at least two sequences which are homologous to one another and/or not homologous.

15. A method in accordance with any one of the preceding claims, characterized in that in step b) and/or in step d) a PCR is carried out which is adapted for the amplification of at least two sequences which are homologous to one another and/or not homologous, with a number of aliquots of the biological sample being made available in step a) which corresponds to the number of the at least two sequences homologous to one another and/or not homologous, and with each aliquot containing the same quantity of the biological material and, in step b) and/or in step d) a PCR is carried out with each of the aliquots in which in each case a primer pair is used, with the primer pairs used in the different PCRs being adapted to amplify the at least two sequences which are homologous to one another and/or not homologous.

16. A method in accordance with any one of the preceding claims, characterized in that the biological sample is amplified prior to carrying out the method step a) with a non-specific PCR and the reaction product which is obtained is, if required, subdivided into the required number of aliquots.

17. A method in accordance with any one of the preceding claims, characterized in that the presence or absence of amplification products takes place by means of gel electrophoresis, by means of a hybridization technique on a DNA array, on a bead system or by means of another optical electrical or electrochemical measurement.

18. A method in accordance with any one of the preceding claims, characterized in that for the determination of the number of different amplification products that are obtained after the amplification reaction the presence or absence of the one sequence or of the at least two sequences which are homologous to one another and/or not homologous and also a second physically and/or chemically measurable parameter of the obtained amplification products is determined.

19. A method in accordance with claim 18, characterized in that the one sequence or at least two sequences homologous to one another and/or not homologous are STR sections and/or VNTR sections and that the length of the amplification products obtained is determined on the second parameter, with the number of the different amplification products obtained corresponding to the number of the amplification products obtained with different lengths.

20. A method in accordance with claim 19, characterized in that the length of the amplification products is determined by capillary electrophoresis.

21. A method in accordance with claim 20, characterized in that the sequence or at least two sequences homologous to one another and/or not homologous are SNP sections and that the sequence of the amplification products obtained is determined as the second parameter, with the number of the different amplification products obtained corresponding to the number of different amplification products with a different sequence.

22. A method in accordance with claim 21, characterized in that the sequence of the amplification products is determined by DNA sequencing or by a hybridization method.

23. A method in accordance with any one of the preceding claims, characterized in that the parameters in the at least one PCR in step b) and/or in step d) are selected such that the relative frequency for a positive amplification reaction for the one sequence or for each of the at least two sequences homologous to one another and/or not homologous is at least substantially the same in each case.

24. A method in accordance with any one of the preceding claims, characterized in that the parameters in the at least one PCR in step b) and/or in step d) are selected such that the relative frequency for a positive amplification reaction for the one sequence or for each of the at least two sequences homologous to one another and/or not homologous amounts to between 0.2 and less than 1, preferably to between 0.4 and 0.6 and also particularly preferably to about 0.5.

25. A method in accordance with any one of the preceding claims, characterized in that an amplification reaction is carried out parallel to the at least one amplification reaction in accordance with step b) and/or step d) under the same conditions with a control sample.

26. A method in accordance with any one of the preceding claims, characterized in that the aliquot of the biological sample in step d) of the method in accordance with patent claim 2 is diluted in a ratio between 1:1 and 1:1000, preferably between 1:1 and 1:100, particularly preferably between 1:1 and 1:10 and quite particularly preferably between 1:1 and 1:2.

27. A method in accordance with any one of the preceding claims, characterized in that, in the characterization of the amplification products, a relative number of alleles of a predetermined sequence is determined.

28. A method in accordance with any one of the preceding claims, characterized in that, for the characterization of the amplification products at least one amplification reaction is carried out under the same conditions as in step b) with a reference sample which preferably contains the same quantity of DNA as the biological sample and which preferably has a known genotype and the number of the different amplification products obtained with this at least one amplification reaction is compared with the number of the different amplification products obtained for only a part of the aliquots or with the number of the different amplification products obtained in step c) less the different amplification products obtained in step e) in accordance with the method of patent claim 2 and/or with the number of the different amplification products obtained for all aliquots or with the number of the different amplification products determined in step e) of the method in accordance with patent claim 2.

29. A method in accordance with any one of the claims 2 to 27, characterized in that the number of the different amplification products obtained in step c) less the different amplification products obtained in step e) and/or the number of the different amplification products determined in step e) is compared with at least one frequency distribution which is also obtained by separate, in each case multiple carrying out of the same at least one amplification reaction as used in step b) and under the same reaction conditions, with the same quantity of starting material having been used or being used in the amplification reactions as named in step a), with at least two different reference samples, wherein the at least two different reference samples each have a known copy number of the predetermined sequence different from each other and also subsequent determination of the number of different amplification products which was or is obtained per reference sample.

30. A method in accordance with any one of the preceding claims, characterized in that for the characterization of the amplification products the amplification products that are obtained are sequenced or subjected to a hybridization method.

31. A method in accordance with any one of the preceding claims, characterized in that for the characterization of the amplification products with one or more dilution stages of the biological sample a multiple determination of at least one PCR is carried out in which at least one allele specific primer pair is preferably used.

32. A method in accordance with claim 31, characterized in that the number of the multiple determinations announce to between 2 and 1000, particularly preferably to between 3 and 100, quite especially preferably to between 4 and 15 and most preferably to between 5 and 10.

33. A kit for the determination of the genotype of one or more individuals from a biological sample which contains nucleic acid of different individuals, in particular for carrying out a method in accordance with any one of the claims 1 to 32, including:
a) at least one primer pair which is adapted to amplify in at least one PCR, one sequence or at least two sequences homologous to one another and/or not homologous, which are included by at least one of the nucleic acids contained in the biological sample,
b₁) a reference sample with a known genotype and preferably with a copy number known with respect to a predetermined sequence and/or
b₂) the result of at least one amplification reaction carried out with a reference sample under the same conditions as described in the protocol in accordance with d), wherein the reaction conditions were so selected that the at least one amplification product arose at a probability between 20% and less than 100% and/or
b₃) at least one frequency distribution which was obtained by separate in each case multiple carrying out of the same at least one amplification reaction with at least two different reference samples carried out under the same reaction conditions as prescribed in the protocol d), with the at least two different reference samples each having a known copy number of a predetermined sequence different from one another, and also subsequent determination of the number of different amplification products obtained per reference sample, and
c) optionally if required PCR buffer and
d) a protocol for carrying out the at least one PCR in a) and, if required, particulars of the dilutions to be effected.

34. A kit in accordance with claim 33, characterized in that the at least one primer pair is adapted to amplify in the at least one PCR one sequence or at least two sequences which are homologous to one another and/or not homologous from the non-coded DNA range, preferably highly polymorphous sequences homologous to one another and/or not homologous, which are particularly preferably selected from the group consisting of STR sequences, VNTR sequences, SNP sequences and any desired combinations hereof.

35. A kit in accordance with claim 33 or 34, characterized in that the at least one primer pair in accordance with a) and/or the protocol in accordance with d) is adapted to amplify in the at least one PCR between 1 and 100, preferably between 2 and 20 and particularly preferably between 5 and 15 sequences which are homologous to one another and/or not homologous.
